(19) United States
(12) Patent Application Publication
(10) Pub. No.: US 2006/0229689 A1
(43) Pub. Date: Oct. 12, 2006

(54) LED therapy device

(75) Inventors: Ronald Leslie Ferguson, Elizabeth, CO (US); Brent Allen Safer, Elizabeth, CO (US)

Correspondence Address: LEYENDECKER LEMIRE & DALEY, LLC, C/O PORTFOLIO IP, P.O. BOX 52050, MINNEAPOLIS, MN, 55402, US

(73) Assignee: LED Technologies, LLC

(21) Appl. No.: 11/399,928

(22) Filed: Apr. 7, 2006

Related U.S. Application Data
(60) Provisional application No. 60/669,844, filed on Apr. 8, 2005.

(51) Int. Cl. A61N 5/06

(52) U.S. Cl. 607/88; 607/91

(57) ABSTRACT

One embodiment of the current LED therapy device comprises at least a left and right LED panel, each panel having a front surface and a back surface, the front surface of the left panel forming an angle relative to the front surface of the right panel of 70° to 170°. Each panel's front surface is generally vertically oriented. The embodiment also includes a detachably coupled pedestal. The pedestal includes a panel attachment section, a stem section, and a base section. The panel attachment section is detachably coupled to the left and right LED panels through a v-shaped slot and flange coupling. The panel attachment section is detachably coupled to the stem section, which, in turn, is also detachably coupled to the base section.

### FIELD OF THE INVENTION

This invention generally relates to light therapy devices.

### BACKGROUND

Since the beginning of time, many of man's therapeutic procedures have focused on physically inserting an item into the body. For example, for thousands of years humans have ingested plants or derivatives of plants in order to alleviate pain and/or reduce unwanted ailments. A specific example of this type of therapy is the ingestion of the herb Yarrow, which has been, and is still, used as a device to stem the flow of blood from open wounds. Additionally, knives and other instruments have also been used as a form of therapy to cut a person to remove unwanted ailments and alleviate pain.

Despite the therapeutic effects of these and other procedures, many of the procedures that are implemented for therapeutic purposes involve unwanted side effects. For example, there may be an extended period of down-time to recover from the effects that an instrument procedure may inflict upon the body. Even if no down-time is associated with the therapy, the specific therapeutic procedure may induce an unwanted side effect such as an introduction of an unwanted condition on the body. For example, Yarrow has the potential to cause dermatitis, an inflammation of the skin, on users.

Even therapeutic procedures that do not involve inserting an item into the body, but involve placing an item upon the body may involve unwanted side effects. Take, for example, the gel-like substance contained in the Aloe plant that has been used for thousands of years to help decrease the time for wounds to heal and to help alleviate the pain, itching, and scratching associated with sunburns and other skin irritations. The plant has also been known to give individuals severe rashes.

To overcome these, and other, side effects from therapeutic procedures, as well as to minimize the time needed to achieve results from an ingestion or application substance, persons have dreamed of implementing a non-invasive, non-ingestive, and non-applicative therapeutic device. For example, the 1960's television show Star Trek used a device the size of a cell phone to send out light rays to close lacerations.

Recent inventions have taken the use of light as a therapeutic device from the realm of science-fiction to an actual science. Based on recent NASA studies such as the “NASA Light-Emitting Diode Medical Program—Progress in Space Flight and Terrestrial Applications” study, persons in the relevant art understand infra-red and near infra-red light of specified wavelength to possess therapeutic qualities. Studies such as the NASA studies explain how exposure to this type of light under certain conditions can alleviate various effects that sun exposure, gravity, pollution, and harsh soaps and chemicals have on the skin.

Prior art LED Devices are known incorporate the belief that infra-red and near infra-red light can provide therapeutic effects to persons exposed to the light under certain conditions. However, these prior art devices do not provide for sufficiently convenient at-home applications for facial use. For example, one known prior art device comprises a hand-held plate better suited for a cosmetic business application. It requires a user to hold the device for the period of facial use, or possibly place the device upon the skin, instead of simply placing one's face in proximity of the device. Likewise, if such a hand-held device is used, it is not likely adapted to provide symmetric and even application of the therapy to all areas of the face.

### SUMMARY OF THE DRAWINGS

FIG. 1 is a front isometric view of an LED therapy device according to one embodiment of the current invention.

FIG. 2 is an exploded front isometric view of an LED therapy device according to one embodiment of the current invention.

FIG. 3 is an isometric view of the back surface of an LED panel according to one embodiment of the current invention.

FIG. 4 is a top view of the front side of an LED panel circuit board according to one embodiment of the current invention.

FIG. 5 is a top view of the back side of an LED panel circuit board according to one embodiment of the current invention.

FIG. 6 is the control diagram for an LED panel circuit board according to one embodiment of the current invention.

FIG. 7 is the top view of the base section of the pedestal according to one embodiment of the current invention.

FIG. 8 is the side view of the stem section of the pedestal according to one embodiment of the current invention.

### DETAILED DESCRIPTION

One embodiment of the current invention comprises two LED panels and a pedestal. Each LED panel includes a front surface and a back surface. The pedestal comprises a base section, stem section, and a panel attachment section. The pedestal may alternatively be referred to as a stand, the base section and stem section may be referred to as a base, and the panel attachment section may be referred to as a mounting bracket.

The base section of the current embodiment typically comprises a top surface, a bottom surface, and one or more side surfaces extending between the outer edge of the top surface and the outer edge bottom surface. The top surface and bottom surface are of generally similar size and shape. The top surface also typically includes a hole, the depth of the hole being generally less than the average height of the one or more side surfaces. The hole is typically of a general oval shape, the hole center being typically located approximately half-way between the top surface center and one of the one or more sides surfaces.

In the current embodiment, the one or more side surfaces gradually increase in height as the distance from the one or more side surfaces to the hole center decreases. The height of the one or more side surfaces is typically such that the top and bottom surfaces have a surface area greater than that of the aggregate of the one or more side surfaces.

The stem section in the particular embodiment comprises a bottom portion, a center portion, and a top portion. The current embodiment's bottom portion is of a generally uniform cylindrical oval-shape adapted to fit into the oval-shaped hole in the top surface of the base section. The stem and hole also contain a fastener securing the stem to the base portion. In the current embodiment, the fastener is at least one flange and one slot, the flange typically being located on the stem and the slot typically placed within the hole. The bottom portion typically ends in a bottom end. The bottom end is typically generally flush against the bottom of the hole.

The center portion of the stem in the particular embodiment is generally comprised of a uniform cylindrical oval-shape whose longitudinal axis is substantially aligned with the longitudinal axis of the bottom portion, and extends from the bottom portion to the top portion of the stem. The top portion of the stem includes a top side. The top portion is of an oval shape and size generally equal to the oval shape and size of the bottom and center portions. However, the longitudinal axis of the top portion is angled relative to the longitudinal axis of the center and bottom portions. The angle between the top side and a vertical plane in the current embodiment is generally less than 20°, although in alternative embodiments the angle may be as great as 70°.

The stem portion top side of the current embodiment includes a fastener and 2 screws. The fastener is adapted to detachably couple the panel attachment portion to the stem section. The screws are adapted to secure the top side to the stem section.

The current embodiment's panel attachment section is comprised of a middle portion, a left portion, and a right portion. Each portion is generally of equal rectangular size, the middle portion left side being integrated with the right side of the left portion and the middle portion right side being integrated with the left side of the right portion. The top and bottom end ends of the left, middle, and right portions are all generally flush. The middle portion includes a fastener that is adapted to detachably couple to the fastener on the stem section top portion top side. The left and right portions are angled relative to the middle portion such that relative angles between the middle portion and the left portion and the middle portion and the right portion, is generally in the range of 125° to 175°. Each of the left and right portions of the particular embodiment also includes 2 slots. Each slot is generally rectangular and create a “v” shape relative to each other, the angle of the two slots relative to each other generally being less than 90°.

In the particular embodiment, one LED panel is detachably coupled to the left portion of the pedestal panel attachment section and a second LED panel is detachably coupled to the right portion of the panel attachment section. The panel attachment portion couples to the left and right LED panels by a pair of flanges on the back side of each LED panel. Each flange pair creates a “v” shape on the LED panel back side that is adapted to mate with the v-shaped slots on the panel attachment left portion and right portion.

In the particular embodiment, by coupling the LED back surface to the left and right panel attachment portions, the LED panel front sides are located next to each other, creating a left panel and right panel. Given the angle of the top side of the stem sections, the front surfaces of the LED panels are also generally vertically oriented. Additionally, given the 125° to 175° degree angle between the front surface of the middle portion of the panel attachment section and the front surface of each left and right portions, the angle created between the front surface of the left LED panel and the front surface of the right LED panel is generally not less than 70° and not more than 170° in the current embodiment. However, it is to be appreciated that the angle between the front surface of the left and right LED panels may be between 90° and 150° or even between 110° and 130°.

In the current embodiment, the LED panel front surface is a uniform color and includes an array of mounted infra-red LEDs. Additionally, dual colurns of non infra-red LEDs are mounted on the front surface. The current embodiment's LED panels also include one or more side surfaces. Included on one of the LED panels'one ore more side surfaces is a power input mechanism, an on/off push button, and a power output. This is the master panel. Power is supplied from a power supply to the power input mechanism. The one or more side surfaces of the other LED panel in the current embodiment includes only a power input. This is the slave panel. Power is supplied from the power output of one of the LED panels to the power input of the other LED panel through a power supply conduit. The particular embodiment's power supply conduit is a power cord coupled to the power input mechanism of one LED panel and the power output of the other LED panel. In alternative embodiments, power may be supplied to more than one slave LED panel from the master LED panel, or power may be supplied to each LED panel from an individual power supply.

The LED panels in the current embodiment also include a circuit board. One of the circuit board LED panels includes a controller and a sound device. The controller includes a timer switch. A 9-minute lighting sequence is controlled by the controller and timer switch. The sound device emits a noise every 1 minute of the 9 minute lighting sequence. The power is released from the power input mechanism to the LEDs by pressing an on/off push button that is connected to an on/off switch.

Terminology:

The terms and phrases as indicated in parenthesis (“ ”) in this section are intended to have the meaning ascribed to them in this Terminology section applied to them throughout this document including the claims unless clearly indicated otherwise in context. Further, as applicable, the stated definitions are to apply, regardless of the word or phrase's case, to the singular and plural variations of the defined word or phrase.

The term “or” as used in this specification and the appended claims is not meant to be exclusive rather the term is inclusive meaning “either or both”.

References in the specification to “one embodiment”, “an embodiment”, “a preferred embodiment”, “an alternative embodiment” and similar phrases mean that a particular feature, structure, or characteristic described in connection with the embodiment is included in at least an embodiment of the invention. The appearances of the phrase “in one embodiment” in various places in the specification are not necessarily all meant to refer to the same embodiment.

The term “couple” or “coupled” as used in this specification and the appended claims refers to either an indirect or direct connection between the identified elements, components or objects. Often the manner of the coupling will be related specifically to the manner in which the two coupled elements interact.

Directional and/or relationary terms such as, but not limited to, left, right, nadir, apex, top, bottom, vertical, horizontal, back, front and lateral are relative to each other and are dependent on the specific orientation of a applicable element or article, and are used accordingly to aid in the description of the various embodiments and are not necessarily intended to be construed as limiting.

As applicable, the terms “about” or “generally” as used herein unless otherwise indicated means a margin of ±20%. Also, as applicable, the term “substantially” as used herein unless otherwise indicated means a margin of ±10%. It is to be appreciated that not all uses of the above terms are quantifiable such that the referenced ranges can be applied.

The term “integrate” or “integrated” as used in this specification and the appended claims refers to a blending, uniting, or incorporation of the identified elements, components or objects into a unified whole.

The term “infra-red” as used in this specification and the appended claims refers to an electromagnetic radiation of a wavelength longer than that of visible light, but shorter than that of microwave radiation, the wavelength of infra-red light being approximately from 750 nm to 1 mm.

The term “LED” and “LEDs” as used in this specification and the appended claims means “Light Emitting Diode” and “Light Emitting Diodes,” respectively.

The term “switch” as used in this specification and the appended claims refers to a device adapted to change the course or flow of electricity by connecting two pieces of metal, called contacts, through an actuator, the actuator being a toggle or dolly, a rocker, a push-button, or any type of mechanical linkage.

### One Embodiment of a LED Therapy Device

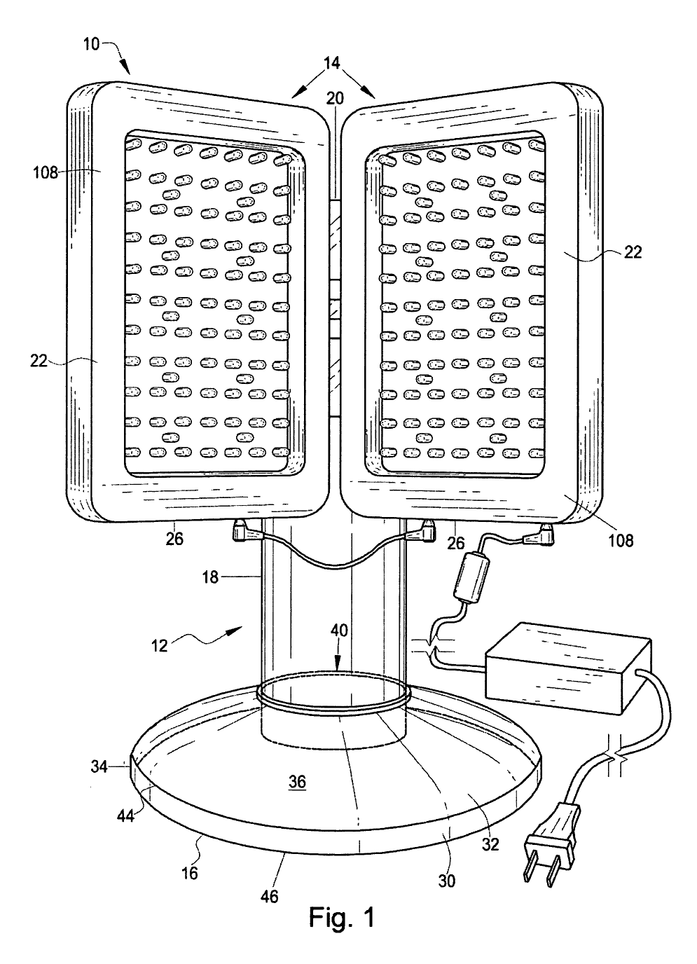

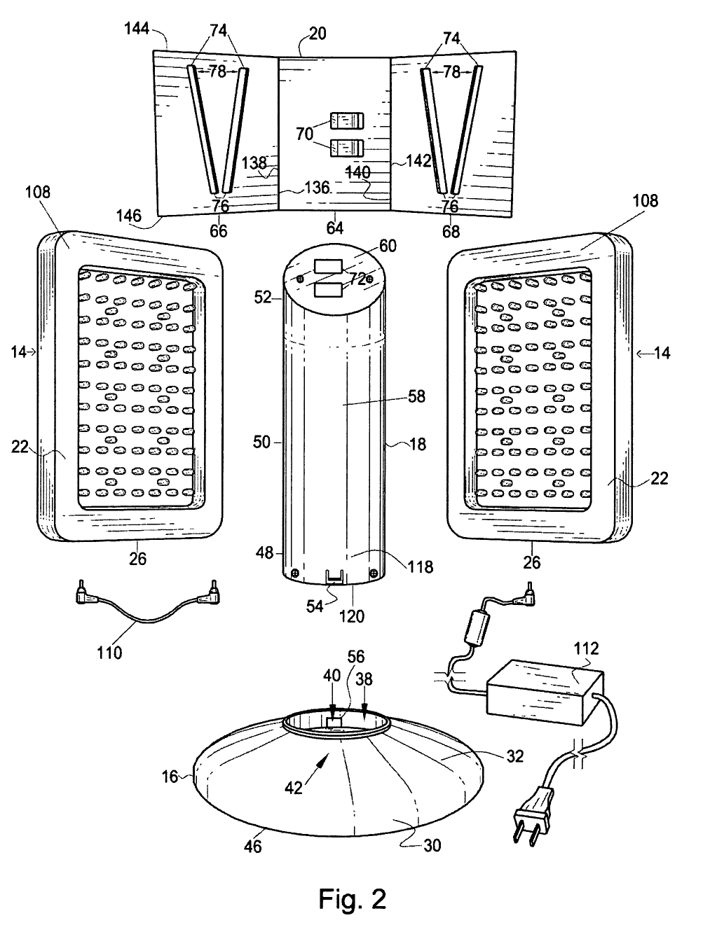

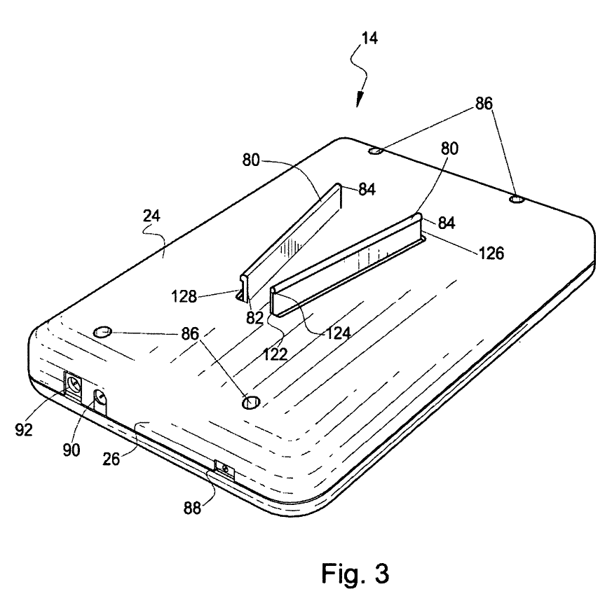

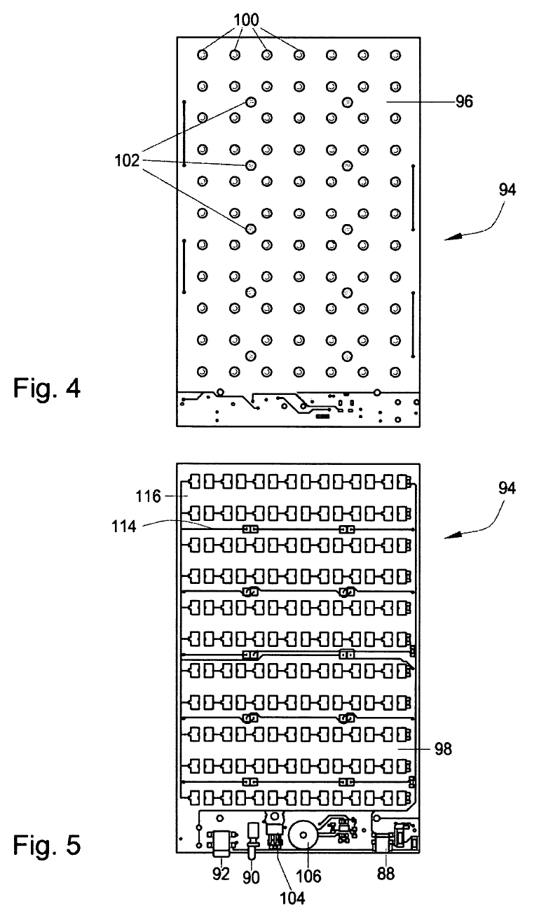

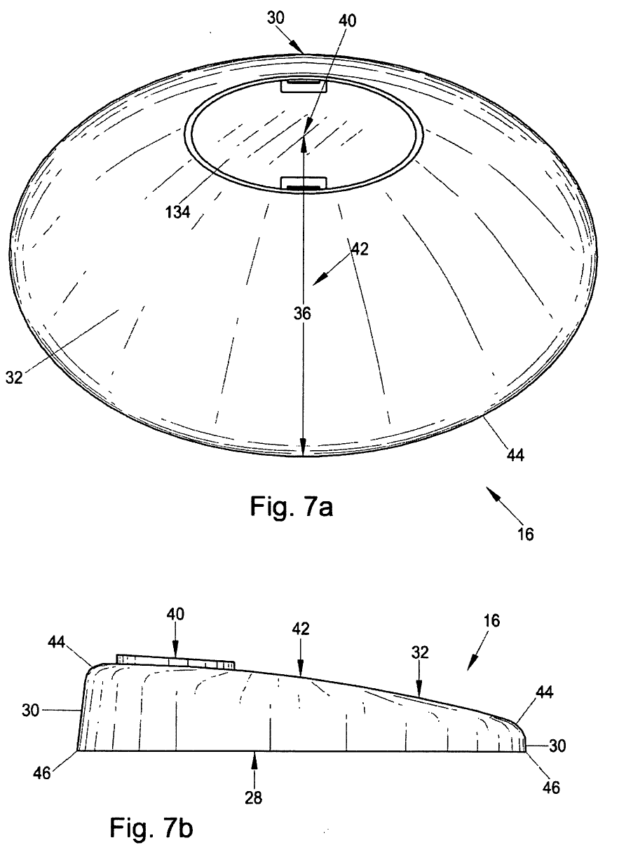

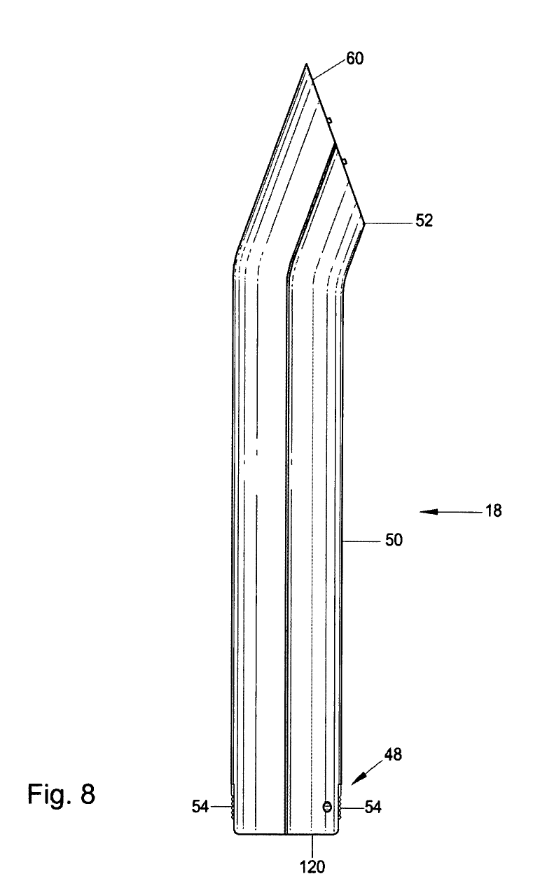

Referring to FIGS. 1 to 8, an embodiment of a LED therapy device 10 is illustrated. The current embodiment is comprised of a pedestal 12 and two LED panels 14. The pedestal is comprised of a detachably coupling base section 16, a stem section 18 and a panel attachment section 20. The LED panels are comprised of a front surface 22 and a back surface 24. The LED panel in the particular embodiment also includes one or more side surfaces 26. Alternative embodiment LED panels may not include one or more side surfaces and alternative embodiment pedestals may not include detachably coupling pedestal sections.

The current embodiment's pedestal sections are typically molded plastic sections. In alternative embodiments, the pedestal sections may be comprised of other material, such as but not limited to, metal sections.

The base section 16 of the current embodiment typically comprises a generally oval-shaped hole 38, a bottom surface 28, a top surface 32 and one or more side surfaces 30. Alternative embodiments may implement a non oval-shaped hole, such as, but not limited to, a circularly-shaped or rectangularly-shaped hole. The center 40 of the hole is generally located on the top surface half-way between the center 42 of the top surface and the one or more side surfaces. The oval hole's minor axis is substantially perpendicular to the one or more side surfaces at the point the minor axis would dissect the one or more side surfaces. The current embodiment's oval hole's major axis dissects the center of the hole. In alternative embodiments, the center of the hole may be located in another position, such as, but not limited to, the center of the top surface. The depth of the hole is such that the bottom 134 of the hole is not as deep as the bottom surface.

The bottom surface 28 of the base section 16 is generally circular in the current embodiment, although alternative embodiments may include bottom surfaces of other geometric shapes. The top surface 32 is generally of the same geometric shape as the bottom surface. However, in alternative embodiments, the top surface may be a different geometric shape than the bottom surface.

The one or more base section 16 side surfaces 30 span between the top surface 32 and the bottom surface 28. The particular embodiment's one or more side surfaces integrates with the outer edge 44 of the top surface and the outer edge 46 of the bottom surface. In the current embodiment, the height 34 of the one or more side surfaces gradually increases as the distance 36 from the edge 44 of the top surface to the center 40 of the hole decreases. The increasing height of the one or more side surfaces generally creates a top surface sloping away from the hole. In alternative embodiments, the height of the one or more side surfaces is generally constant. For example, if the center of the hole is located in the center 42 of the top surface, the height of the one or more side surfaces may be generally constant. Additionally, there may be embodiments that do not incorporate the one or more side surfaces. In such an embodiment, the top surface edge 44 will contact the bottom surface edge 46.

The stem section 18 of the particular embodiment's pedestal is comprised of a bottom portion 48, a center portion 50, and a top portion 52. The bottom portion is comprised of an outside surface 118, a bottom end 120, and a fastener 54. The bottom portion is adapted to fit securely within the hole 32 on the top surface 32 of the base section. The fastener ensures the bottom portion is secure within the hole, providing additional stability. In the current embodiment, the fastener is a flange-like protrusion from the stem section. The flange inserts into a slot 56 in the hole. However, the fastener may be any other type of detachably coupling device as well, such as, but not limited to, a device that snappably connects to the hole. Once secure, the bottom end is substantially flush with the bottom 134 of the hole.

The bottom portion 48 is typically integrated to the center portion 50. The outside surface 58 of the center portion in the current embodiment is of generally the same size and shape as the outside surface 118 of the bottom portion. Therefore, in the current embodiment, the bottom portion and the center portion generally create an elliptical cylinder with a single longitudinal axis. In alternatives, the center portion may be of an expandable nature, thereby allowing the stem section to expand in height. Other alternatives may allow the center section to bend and twist or the center portion's outside surface may be of a shape different than the bottom portion's outside surface. In other alternatives, the center portion may not create an elliptical cylinder or may be a cylinder that does not possess the same longitudinal axis as the bottom portion.

In the current embodiment, the top portion 52 of the stem section 18 is also typically an oval-shaped cylinder and includes a top side 60. However, in the current embodiment, the top portion is angled relative to the bottom 48 and center 50 portions. The top portion is angled towards the semiminor axis of the oval hole that is further away from the one or more side surfaces. In an alternative embodiment where the semiminor axes are of equal distance from the one or more side surfaces, the angle may be towards either of the semiminor axis. In the current embodiment, therefore, the top side of the top portion of the stem section is typically not parallel to the bottom end 120 of the bottom section 48.

The angle between the top side 60 and a vertical plane in the current embodiment is generally less than 20°, although in alternative embodiments the angle may be as great as 70°. In additional alternatives, the top side may be substantially vertical, therefore there may be no angle. In other alternatives, the angle of the top side and the vertical plane is between 30° and 50°. The length of the stem section in the current embodiment is such that when the LED panels are coupled to the panel attachment section, the panels do not come in contact with the top surface of the base section.

The pedestal 12 panel attachment section 20 is comprised of a middle portion 64, a left portion 66, and a right portion 68. Each portion is generally of equal rectangular size, the middle portion left side 136 being integrated with the right side 138 of the left portion and the middle portion right sidel40 being integrated with the left side 142 of the right portion. The top 144 and bottom 146 end ends of the left, middle, and right portions are all substantially flush. In alternative embodiments, each portion may be of unequal rectangular sizes, or may not be rectangular at all. Additionally, the portions may not be integrated or possess flush ends.

The panel attachment section 20 in the current embodiment is detachably coupled to the stem section 18 trough a fastener included in the middle portion 64. The typical fastener includes two flange-like devices 70 that mate to two holes 72 on the top side 60 of the stem section 18. The left 66 and right 68 portions of the panel attachment section of the particular embodiment create an angle with the middle portion between 125° to 175°. Alternatively, the angle created is 135° to 165°, or from 140° to 160°.

Each of the left 66 and right 68 portions of the panel attachment section 20 include two slots 74. The slots are generally vertically oriented and create an upwardly-opening “v,” on each of the left and right portions. Therefore, the relative distance between the slots gradually increases from the lower ends 76 of the slots to the upper ends 78 of the slots. In alternative embodiments, there may not be two slots that are arranged to form a v-shape, but the slots may be a single slot in the shape of an upwardly facing “v.” Also, in alternatives, the dual or single slot portions may comprise a downwardly facing v, or any other directionally opening v-shaped slot. In other alternatives, the slots may not be v-shaped.

In the particular embodiment, one set of the v-shaped slots 74 mate with flanges 80 located on the back surface 24 of one LED panel 14. The two sets of v-shaped slots typically mate with two sets of flanges integrated into two LED panels. One reason the LED panels are detachable is so that a user can use the LED panels as hand-held devices. The slot/flange v-shape of the LED device is to enable a user to use the flanges as a handle when detaching an LED panel from the panel attachment section. Therefore, the flanges may be described as “handles.” In alternative embodiments, there maybe more than two LED panels. If there are more than two LED panels, each LED panel back surface may not include two flanges. Other embodiments may implement more panel attachment portions than a left 66, right 68, and middle 64 portion. Still further embodiments may include less than a left, right, and middle panel portion.

Each flange 80 in the particular embodiment is arranged to create a v-shape flange corresponding to the slots 74 that are arranged to form a v-shape. Typically, the flanges are arranged vertically on the back surface 24 of the LED panel 14. The relative distance between a first section 150 and a second section 152 top end 126 is greater than the distance between the bottom ends 128 of each flange. Each flange may also be oriented to create a downward-opening “v,” or any other directionally-opening “v” to correspond to the directional opening of the slots in other embodiments.

The flanges 80 are also comprised of a first portion 82 and a second portion 84. The first portion is generally perpendicularly integrated to the back surface 24 of the LED panel 14 at a first portion proximal end 122. In alternative embodiments, the first portion is not integrated into the back surface, but is coupled to the back surface through a coupling device such as, but not limited to, a sliding slot or a snapping slot. Integrated to the flange first portion at a first portion distal end 124 is the second portion. The second portion is typically shorter and wider than the first portion. In alternative embodiments, the second portion is not shorter than the first portion, but may be longer than, or equal to, the length of the first portion.

The back surfaces 24 of the LED panels 14 on the particular embodiment are generally rectangular. Additionally, the back surface includes four screws 86 attaching the back surface to the rest of the LED panel. In alternative embodiments, the screws do not attach the back surface to the rest of the LED panel or the back surface may contain more or less than 4 screws, or the back surface may be comprised of no screws. Alternative embodiments may attach the back surface of the LED panel to the rest of the LED panel in a different manner, such as, but not limited to, implementing a snappable connection or a molded integration.

The back surface 24 of the LED panels 14 in the current embodiment are typically comprised of a plastic material. However, alternative embodiments may consist of materials such as, but not limited to, a composite material.

One of the LED panel's 14 one or more side surfaces 26 includes a power output mechanism 88, an on/off push button 90, and a power supply input mechanism 92. The other LED panel's one or more side surfaces only includes a power supply input mechanism. In the current embodiment, it is typically the left LED panel whose one or more side surfaces only includes the power input mechanism. In alternative embodiments comprising more than one left and one right LED panel, more than one LED panel's one or more side surfaces may only comprise the power input mechanism and more than one LED panel's one or more side surfaces may comprise a power output mechanism, an on/off push button and a power input mechanism. Alternative embodiments may also be comprised of only one LED panel.

In the current embodiment, the power output 88 on the one or more side surfaces 26 of one LED panel 14 supplies power to the power supply input mechanism 92 of the other LED panel. The LED panel including the power output may be referenced as the master panel and the LED panel not including the power output may be reference as the slave panel. The power is transmitted between the panels through a power conduit 1 10 such as, but not limited to, a power cord. Power is supplied to the power supply input mechanism of the LED panel containing the power supply output and the on/off switch through a power supply 112 that is connected to an external power supply, such as, but not limited to, a wall outlet having a range of 100 volts to 240 volts. In alternative embodiments, the power supply 112 may be connected to a power supply such as an in-home power generator.

The LED panel 14 of the current embodiment may also be used as a hand-held therapy device. Typically, the LED panel including the power output 88 is the panel used as the hand-held device. The LED panel's flanges may be used as an embedded handle. In such a configuration, the user typically disconnects the power conduit from the power output and uncouples the LED back surface flanges from the panel attachment section slots in order to use the device as a hand-held.

The particular embodiment's LED panels 14 also contain circuit boards 94. The circuit boards are comprised of a front side 96 and a back side 98. The front side is comprised of a uniform color, typically black. The circuit board front side in alternative embodiments may be comprised of a reflective color such as, but not limited to, a mirror. Alternative embodiments' front sides may also be comprised of a non-black, non-mirrored uniform color, such as, but not limited to, a gray color, or even a non-uniformly colored front side.

The current embodiment's circuit board 94 front side 96 is also comprised of a plurality of infra-red LEDs 100 and non infra-red LEDs 102 mounted thereon. Mounting LEDs directly to the front side of the circuit board allows the front side of the circuit board to be used as the front side of the LED panel, cutting down on cost. The current embodiment is comprised of 7 columns of infra-red LEDs, each column including 11 rows. Alternative embodiments may include additional infra-red LEDs by including an additional row or rows or an additional column or columns. Alternative embodiments may also include less LEDs by including a lower number of columns or rows of infra-red LEDs. Additional embodiments may employ near infra-red LEDs in addition to, or in place of, any of the infra-red LEDs. Also mounted on the front side of the particular embodiment are two columns of non infra-red LEDs, containing 5 rows each. The current embodiment uses the specific configuration and number of LEDs in order to provide the level of infra-red light necessary to provide the user with the therapeutic effect. Alternative embodiments may includes less or more non infra-red LEDs.

The infra-read LEDs in the particular embodiment produce approximately 2 J/cm² of light at a wavelength of approximately 880 nm. Studies have shown that this infra-red light at this wavelength and power level may have a therapeutic affect on skin. Non infra-red LEDs produce light at a wave length of approximately 660 nm. Non infra-red light of this wavelength is used because (i) light of this wavelength is within the visible spectrum, so the user knows the device is powered on and working; and (ii) the wavelength of light may possess therapeutic qualities for skin. Alternative embodiment LEDs may produce light at wavelengths of anywhere between approximately 660 nm and 880 nm at power levels above 1.5 J/cm² with power levels of about 1.8-4 J/cm² being more preferable. At these wavelengths and power levels the LEDs may also possess therapeutic qualities. It is further contemplated that in other variations LEDs may be used that produce infrared light in any part of the infrared spectrum. Further, any non-infrared LEDs that produce visible light may be utilized in place of the 660 nm LEDs for informing the user that the unit is currently active and operational.

In the current embodiment, mounted on the back side 98 of the circuit boards 94 is a power supply input mechanism 92. Also mounted on the back side of the circuit boards are traces 114 etched from copper sheets and laminated onto a non-conductive substrate 116. On one of the boards is further mounted an on/off switch 132, a controller 104, a sound device 106, and a power supply output 88. In alternative embodiments, more than two LED panels 14, and therefore more than two circuit boards may be employed. In such a embodiment, or even in the current embodiment, one or more of the components mounted on the one of the circuit boards may be mounted on a circuit board only containing the power supply input mechanism in the current embodiment.

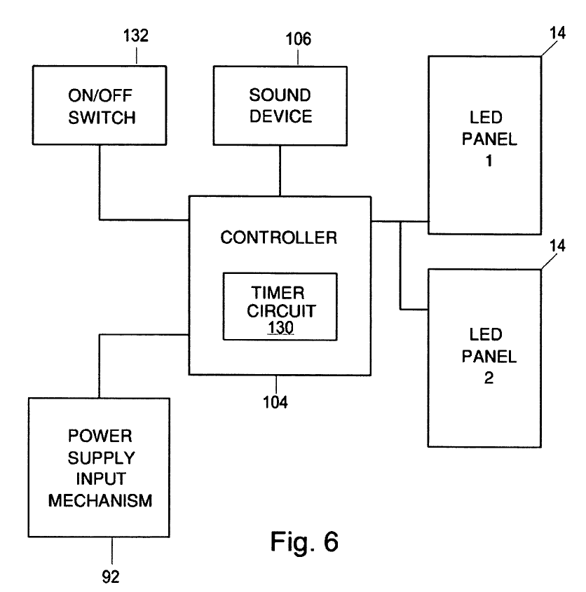

As shown in FIG. 6, the controller 104 of the LED panel 14 circuit board 94 is adapted to receive power from the power supply input mechanism 94. The controller in the current embodiment is a microprocessor. Included in the controller is a timer circuit 130. In alternative embodiments, the timer circuit may be a separate microprocessor. Also in other embodiments the controller and/or the timer circuit may be a read-only memory device or may be a flash memory device.

Upon power being supplied to the controller 104 through the power supply input mechanism 92, the on/off switch 132 must be turned on in order for the controller to signal the timer circuit 130 to initiate a timing sequence. The on/off switch is typically connected to a push-button 90 located on the one or more side surfaces. The timing sequence in the current embodiment is 9 minutes. A 9 minute timing cycle is used in order to provide a user with the amount of light necessary for the therapeutic effect of the light to take effect on the skin at the current LED configuration. Alternative embodiments may have longer or shorter timing sequences or may not employ a push-button. In some alternative, the timing sequence may be different because the LED wavelength and power is different than the current embodiment.

In the current embodiment, the sound device 106 may also be called a buzzer. The buzzer emits a noise every 60 seconds to let the user know that device is still working and sending power to the LEDs. The noise typically lasts for approximately 1 second. In alternatives, the buzzer may “buzz” at intervals more or less than 1 minute, and may emit a noise for longer than 1 second.

During the timing sequence, the controller 104 allows power to be sent to the LED panels 14. The timer circuit 130 informs the controller when the timing sequence is complete and the controller ceases to power to be sent to the LED panels 14.

The current embodiment also comprises an LED panel 14 front surface 22. The front surface of the LED panel is comprised of a border 108 surrounding the circuit board 94 front side 96. The circuit board front side is recessed from the border. The border, and the one or more side surfaces 26 in the particular embodiment are comprised of a plastic material. In alternative embodiments, the border and one or more side surfaces are comprised of material such as, but not limited to, a metal or composite material. It is to be appreciated that the size of the LED panels 14 are such that the panels fit in panel attachment section 20.

### One Method of Using an Embodiment of a LED Therapy Device

One method of using an embodiment of an LED therapy device 10 includes setting up the device. Setting up the device includes placing the pedestal 12 base section 16 on a generally flat surface. The flat surface may be a surface such as, but not limited to, a desktop or countertop. Once the base section is in place, the stem section 18 bottom portion 48 is coupled to the base section. To ensure the stem section is secure, the stem section's flange-like device 54 is tested to ensure it is placed within the slot 40 located on the base section. Upon verification that the stem section is secure, the panel attachment section 20 is secured to the stem section.

Once the pedestal 18 is set up in the current embodiment, the LED panels 14 are placed onto the panel attachment section 20 by placing the flanges 80 on the LED panel back surface 24 into the slots 74 located on the panel attachment section. The LED panels are then connected with a power conduit 110 and power is supplied one of the LED panels through a power supply 112.

Power is sent to the infra-red LEDs 110 and the non infra-red LEDs 102, and the timing sequence is initiated, by a user switching on the on/off switch 90. The user then places the part of the body the user wishes to provide therapy to between the left and right LED panels 14. For example, the user may place his or her face between the LED panels such that the person's nose may nearly touch the border 108 of the left and right LED panels. In an alternate use of the LED panels, the user may disconnect the power conduit 110 from one of the left and right LED panels, remove the LED panel from the panel attachment section 20, and place the front surface 22 of the LED panel proximate to the area of the body the user wishes to provide therapy to.

When the on/off switch 90 is switched on, the buzzer 106 emits a sound after every 60 seconds has elapsed during the timing sequence. After a nine-minute lighting sequence in the current embodiment, the timing cycle is stopped and power ceases to be sent to the LED panels 14. The user may then disassemble the LED therapy device 10, or may keep the device assembled and ready for use during the next timing sequence.

### Alternative LED Therapy Device Embodiments

The various embodiments and variations thereof illustrated in the accompanying Figures and/or described above are merely exemplary and are not meant to limit the scope of the invention. It is to be appreciated that numerous variations of the invention have been contemplated as would be obvious to one of ordinary skill in the art with the benefit of this disclosure.

We claim:

1) An LED therapy device comprising:
at least a left and right LED panel, each panel having a front surface and a back surface, the front surface of the left panel forming an angle relative to the front surface of the right panel of 70° to 170° and being generally vertically oriented;
a pedestal including a panel attachment section, a stem section, and a base section, the left and right LED panels being detachably coupled to the panel attachment section, and the stem section being coupled to the base section and the panel attachment section.

2) The LED therapy device of claim 1 wherein, each LED panel further includes a circuit board, the circuit board having a front side and a back side,
the front side being of general uniform color and including a plurality of LEDs mounted thereon, and
the back side (i) including a power supply input mechanism, and (ii) adapted to route power to the plurality of LEDs.

3) The LED therapy device of claim 2 wherein, the plurality of LEDs comprises an array including a plurality of infra-red LEDs and a plurality of non infra-red LEDs.

4) The LED therapy device of claim 3 wherein, one of the left or right LED panels comprises a first LED panel and the other of the left or right LED panels comprise a second LED panel, the back side of the circuit board of the first LED panel further including an on/off switch, a controller, a sound device, and a power supply output.

5) The LED therapy device of claim 4 wherein, the power supply output is adapted to supply power to the power supply input mechanism of the second circuit board of the second LED panel through a power supply conduit.

6) The LED therapy device of claim 1 wherein, one of the panel attachment section and both the left and right LED panel back surfaces includes a flange and the other of the panel attachment section and the left and right LED panel back surfaces includes a slot adapted to receive the flange therein.

7) The LED therapy device of claim 6 wherein, the flange and slot are v-shaped.

8) The LED therapy device of claim 7 wherein, both the left and right LED panel back surfaces include the v-shaped flange, the v-shape flange generally vertically oriented, having a left section, a right section, a top end, a bottom end, a first portion, and a second portion,
the bottom end of the left and right flange sections having a relative distance between less than the top end of the left and right flange sections,
the flange first portion being substantially perpendicularly coupled to the back side of the one or more LED panels at a first portion proximate end, and
the flange second portion (i) integrated with the first portion at a first portion distal end, and (ii) being wider than the flange first portion.

9) The LED therapy device of claim 1, wherein one of the left or right LED panels further includes one or more side surfaces, the one or more side surfaces having a power input, an on/off switch, and a power output.

10) An LED therapy device comprising:
a pedestal including 2 or more sections adapted to be detachably coupled,
at least one LED panel including a front side, a power supply input, an on/off switch, a buzzer, a controller, and a timing circuit,
the LED panel being detachably coupled to the pedestal, and
the front side (i) being of generally uniform color and (ii) including an array of mounted infra-red LEDs.

11) The LED therapy device of claim 10 wherein,
power is supplied to the power supply input,
the timing circuit is adapted to initiate a 9-minute lighting cycle upon the switching on of the on/off switch.

12) The LED therapy device of claim 11 wherein, the buzzer is adapted to emit a noise approximately every 60 seconds during the 9-minute lighting cycle.

13) The LED therapy device of claim 10, wherein the array of mounted infra-red LEDs consists of 7 columns and 11 rows.

14) The LED therapy device of claim 13, wherein the wavelength of the light from the infra-red LEDs is generally 880 nm and the power produced by the infra-red LEDs is generally 2 J/cm².

15) The LED therapy device of claim 13, further including a plurality of non infra-red LEDs, the non infra-red LEDs being arranged in a first and a second substantially vertical column, each column having 5 non infra-red LEDs.

16) The LED therapy device of claim 15, further including an LED array left end and an LED array right end, the first non infra-red LED column being mounted to the right of the second infra-red LED column nearest the LED array left end and the second non infra-red LED column being mounted to the left of the second infra-red column nearest the LED array right end.

17) The LED therapy device of claim 10, wherein at least one LED panel comprises a left and right LED panel, power being supplied to one of the left or right panels through a power supply and power being supplied to the other of the left or right panel through a power conduit between the two panels, power supply adapted to receive power from an outlet having a voltage range of approx 100 volts-240 volts,

18) An LED therapy device comprising:
a pedestal including at least two v-shaped slots, and
left and right LED panels, each panel including a front side and a back side, the front side comprising an array of mounted LEDs, the back side including a v-shaped flange adapted to detachably couple the LED panel to the pedestal.

19) The LED therapy device of claim 18 wherein, the v-shaped flange comprises two generally vertically oriented flange sections, having a top end and a bottom end, each flange section being spaced from the other flange section a first distance at the bottom end and a second distance at the top end, -the second distance being greater than the first distance.

20) The LED therapy device of claim 19, each flange section comprises a first portion and a second portion,
the first portion being substantially perpendicularly coupled to the back side of the one or more LED panels at a first portion proximate end, and
the second portion (i) integrated with the first portion at a first portion distal end, and (ii) being wider than the first portion.
